(19) United States
(12) Patent Application Publication
(10) Pub. No.: US 2005/0227300 A1
(43) Pub. Date: Oct. 13, 2005

(54) Use of alpha-tubulin acetylation levels as a biomarker for protein deacetylase inhibitors

(76) Inventors: Peter Wisdom Atadja, Action, MA (US); Sjouke Hoving, Basel (CH); Harry Towbin, Allschwil (CH); Heather Brownlee Walker, South Orange, NJ (US); Markus Wartmann, Riehen (CH); Lakshmi Yeleswarapu, Millburn, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/497,333

(22) PCT Filed: Dec. 6, 2002
(86) PCT No.: PCT/EP02/13873

§ 371 Date: May 19, 2005

Related U.S. Application Data
(60) Provisional application No. 60/338,231, filed on Dec. 7, 2001.

(51) Int. Cl. G01N 33/574; A61K 49/00

(52) U.S. Cl. 435/7.23; 424/9.2

(57) ABSTRACT

The invention relates to a novel method for evaluating the antiproliferative activity of protein deacetylase inhibiting compounds and microtubule interacting agents, as well as a method for screening for compounds that inhibit cell growth or growth of tumors. The invention additionally provides a method for monitoring the progress of treatment against cellular growth or the growth of tumors.

This invention relates to the use alpha-tubulin acetylation levels as a biomarker for protein deacetylase inhibition and as a biomarker for the activity of microtubule interacting agents, particularly as a method to identify compounds having antiproliferative activity.

### BACKGROUND

Protein deacetylase inhibiting compounds, such as histone deacetylase (HDAC) inhibiting compounds, and microtubule interacting agents, are being investigated as therapeutic agents for the treatment of proliferative diseases. It has been found that when the enzymatic activity of a protein deacetylase, such as histone deacetylase, is interrupted, increased acetylated alpha-tubulin is induced and accumulates in the cell. Acetylation of alpha-tubulin usually occurs on Lysine 40. It has further also been found that microtubule interacting agents induce acetylation of alpha-tubulin. It is important to have biomarkers in order to identify and measure the activity of protein deacetylase inhibiting compounds or of micotubule interacting agents, for a variety of applications, such as the screening of compounds and diagnostic use.

### SUMMARY

The present invention relates to the use of alpha-tubulin acetylation as a biomarker for protein deacetylase activity, including histone deacetylase activity. This invention Is based on the discovery that accumulation of acetylated alpha-tubulin in cells is induced by a protein deacetylase inhibitor and is not a cell cycle dependent event. Thus, measurement of alpha-tubulin acetylation is particularly useful to identify compounds that modulate protein deacetylase activity and as a diagnostic method for identifying patients that may benefit from treatment with a protein deacetylase inhibiting compound and/or monitoring the progress of treatment. Likewise, the present invention also relates to the use of alpha-tubulin acetylation as a biomarker for the activity of microtubule interacting agents.

### DETAILED DESCRIPTION

The present invention relates to aspects of the discovery that alpha-tubulin acetylation is useful as a biomarker for protein deacetylase activity and as a biomarker for the activity of microtubule interacting agents.

In one aspect, the present invention relates to a method of screening a compound for antiproliferative activity, which comprises contacting mammalian cells with the compound and detecting an increased level of acetylated alpha-tubulin relative to a control.

Generally, the mammalian cells are a cell line established from a subject having a proliferative disease, especially when the mammalian subject is a human and the proliferative disease is cancer. Especially useful cell lines are established from human cancers such as a lymphoma, a myeloma, such as especially multiple myeloma, a leukemia, a small cell lung carcinoma, a non-small cell lung carcinoma, an osteosarcoma, a breast carcinoma, a prostrate cancer or a colon cancer or a rodent cell line such as a contact inhibited mouse fibroblast cell line.

Generally, the cells are in a cell culture and various experiments are employed to screen potential compounds. In particular, the cell culture is treated with a compound and cultivated. The culture is then assayed to determine the expressed level of acetylated alpha-tubulin. Induction of acetylated alpha-tubulin in the cell culture by a compound indicates that the compound possesses activity as a protein deacetylase inhibitor.

Cell lines that are particularly useful for screening compounds in a cell culture include, for example, HCT 116 colon carcinoma cells (ATCC No.: CCL-247), H1299 lung carcinoma cells (ATCC No.: CRL-5803), A549 non-small cell lung cancer cells (ATCC No.: CCL-185), MDA-MB-435 non estrogen-dependent breast adenocarcinoma cells, PC-3 prostate cancer cells (ATCC No.: CRL-1435), DU145 prostate cancer cells.

In an alternative method, the mammalian cells are implanted into a non-human mammalian host. Generally, this will be a xenotransplant, for example where a human tumor cell line is Implanted into a rodent, such as a mouse, by methods known in the art. The compound to be screened is then administered to the host by a suitable method, for example systemically or locally, by oral administration, injection or another route, according to a dosage regimen.

Often the compound is administered multiple times over a period of time, for example three, four or five times a week for one, two, three, four or more weeks. According to the present invention, the progress of the disease, and thus the efficacy of the compound, Is monitored by measuring alpha-tubulin acetylation levels in tumors from the host animal.

Levels of expression of acetylated alpha-tubulin are assayed in a biological sample, e.g., cell lysate, tissue lysate or white blood cell lysate, by known methods, including immunoassays and electrophoresis assays. For example, acetylated alpha-tubulin-specific antibodies are used in a standard immunoassay format to measure acetylated alpha-tubulin levels. ELISA (enzyme linked immunosorbent assay) type assays and conventional Western blotting assays using e.g. monoclonal antibodies are also utilized to make direct determination of the induction and accumulation of the acetylated alpha-tubulin as a biomarker protein.

Antibodies specific to acetylated alpha-tubulin are produced in accordance with known immunization methods.

The acetylated alpha-tubulin level is also measured by two-dimensional (2-D) gel electrophoresis. 2-D gel electrophoresis Is known in the art and typically involves isoelectric focusing (IEF) along a first dimension followed by SDS-PAGE (sodium dodecyl sulphate-polyacrylamide gel electrophoresis) along a second dimension. The resulting electropherograms are analyzed, for example, by immunoblot analysis using antibodies. Suitable antibodies can be produced as discussed above or obtained from a commercial source. For the immunoblotting analysis, the antibody does not have to be specific to acetylated alpha-tubulin and can be an antibody that is reactive to any form of alpha-tubulin because unacetylated alpha-tubulin and induced acetylated alpha-tubulin are easily separated by IEF.

The inventive screening method is useful for identifying compounds that possess therapeutic activity against a proliferative disease. In particular, the compounds identified according to the inventive screening method are protein deacetylase Inhibiting compounds, such as tubulin deacetylase inhibiting compounds, especially alpha-tubulin deacetylase inhibiting compounds, and histone deacetylase inhibiting compounds, with an activity that is qualitatively like that of trapoxin, trichostatin, N-hydroxy-3-[4-[[[2-(benzofur-3-yl)-ethyl]-amino]methyl]phenyl]-2E-2-propenamide (HDAC-1), N-hydroxy-3-[4-[[(2-hydroxyethyl)[2-(1H-indol-3-yl)ethyl]-amino]methyl]phenyl]-2E-2-propenamide (HDAC-2), N-hydroxy-3-[4-[[[2-(1H-indol-3-yl)ethyl]-amino]methyl]phenyl]-2E-2-propenamide (HDAC-3) and N-hydroxy-3-[4-[[[2-(2-methyl-1H-indol-3-yl)-ethyl]-amino]methyl]phenyl]-2E-2-propenamide (HDAC-4), and other protein deacetylase inhibiting compounds.

Histone deacetylase inhibiting compounds are typically those which have an IC₅₀ of less than 2 μM, especially of less than 500 nM, and most preferably of less than 100 nM in the histone deacetylase inhibition assay described in Example B2 of WO 02/22577. Histone deacetylase inhibiting compounds, including HDAC-1, HDAC-2, HDAC-3 and HDAC-4 mentioned in the preceding paragraph, and the preparation thereof are described e.g. in WO 02/22577 published on March 21, 2002 and are herewith incorporated by reference.

The present invention also provides a method for monitoring therapeutic efficacy of an active compound which inhibits or regulates protein deacetylase activities. According to the present invention, alpha-tubulin acetylation is used as a clinical marker to monitor the efficacy of a protein deacetylase inhibitor compound on a patient. When a protein deacetylase inhibiting compound has an efficacious effect on the proliferative condition, a biological sample, for example, serum or tissue, from the patient will show that the patient has an elevated level of acetylated alpha-tubulin, especially in the target cells. Similarly, the measurement of acetylated alpha-tubulin levels is used to optimize the dosage and the regimen of an active compound by monitoring the induction and accumulation of acetylated alpha-tubulin in biological samples from the subject.

Accordingly, the screening method of the present invention can be used to find a therapeutically effective compound and/or to find a therapeutically effective amount or regimen for the selected compound, thereby individually selecting and optimizing a therapy for a patient. Factors for consideration in this context include the particular condition being treated, the particular mammal being treated, the clinical condition of the individual patient, the site of delivery of the active compound, the particular type of the active compound, the method of administration, the scheduling of administration, and other factors known to medical practitioners. The therapeutically effective amount of an active compound to be administered will be governed by such considerations, and is the minimum amount necessary to prevent, ameliorate, or treat the disease. Such amount is preferably below the amount that is toxic to the host or which renders the host significantly more susceptible to infections.

In addition, the invention further relates to non-histone protein deacetylases, such as tubulin deacetylases, especially alpha-tubulin deacetylase, as novel therapeutic target family, especially as a target for therapeutic agents useful for the treatment of proliferative diseases.

The invention provides compositions and methods for treating or preventing proliferative diseases, especially cancer, in human and veterinary patients, compositions and methods for screening a library of agents for pharmacological activity in regulating cell proliferation and/or cell differentiation, compositions and methods for modulation of a transformed cell phenotype in vitro, including use in bioprocess control and as commercial laboratory reagents. This invention further relates to a novel method for evaluating the antiproliferative activity of compounds which induce alpha-tubulin acetylation.

Thus, the present invention includes a method of evaluating a response by a mammalian subject to a protein deacetylase inhibiting compound which comprises measuring the level of acetylated alpha-tubulin in cells of the subject and comparing it to the level prior to administration of the protein deacetylase inhibiting compound, especially wherein the mammalian subject is a human who has a proliferative disease such as especially a cancer. In a specific embodiment of the aforementioned method, the levels of acetylated alpha-tubulin are measured ex vivo, i.e. outside the body of the mammalian subject. This can be done by taking a biological sample from the mammalian subject and assaying the level of expression of acetylated alpha-tubulin in the biological sample according to known methods such as immunoassays and electrophoresis assays.

The present invention further includes a method of diagnosing a proliferative disease susceptible to treatment with protein deacetylase inhibiting compounds in a mammalian subject, which comprises measuring in cells of the subject that exhibits the proliferative disease an increased level of acetylated alpha-tubulin compared to the level prior to administration of the protein deacetylase inhibiting compound. In a specific embodiment of this method, the levels of acetylated alpha-tubulin are measured ex vivo (see definition of “ex vivo” in the preceding paragraph).

The present invention also provides a method of treating a proliferative disease in a mammalian subject, which comprises administering a protein deacetylase inhibiting compound to the subject that exhibits the proliferative disease and measuring in cells of the subject an increased level of acetylated alpha-tubulin compared to the level prior to administration of the protein deacetylase inhibiting compound.

The present invention further includes a method of treating a proliferative disease in a mammalian subject, which comprises measuring the level of acetylated alpha-tubulin in cells from the subject that exhibits the proliferative disease and administering a protein deacetylase inhibiting compound to the subject if the level of acetylated alpha-tubulin is lower than that exhibited by normal cells of the same type, in particular wherein the protein deacetylase inhibiting compound is a histone deacetylase inhibiting compound, especially a compound selected from N-hydroxy-3-[4-[[[2-(benzofur-3-yl)-ethyl]-amino]methyl]phenyl]-2E-2-propenamide, N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(1H-indol-3-yl)ethyl]-amino]methyl]phenyl]-2E-2-propenamide and N-hydroxy-3-[4-[[[2-(2-methyl-1H-indol-3-yl)-ethyl]-amino]methyl]phenyl]-2E-2-propenamide, or a pharmaceutically acceptable salt thereof.

Surprisingly, it was also found that microtubule interacting agents, which are well known for their antiproliferative and antitumor properties, induce acetylation of alpha-tubulin. The present invention therefore also relates to the use of alpha-tubulin acetylation as a biomarker for the activity of microtubule interacting agents.

The term “microtubule interacting agents” relates to microtubule stabilizing and microtubule destabilizing agents including, but not limited to the taxanes paclitaxel and docetaxel, the vinca alkaloids, e.g., vinblastine, especially vinblastine sulfate, vincristine, especially vincristine sulfate, and vinorelbine, discodermolide and epothilones, such as epothilone B and D. When administered to a mammalian subject, such as e.g. a human, docetaxel, vinblastine sulfate and vincristine sulfate, can be administered, e.g., in the form as it is marketed, e.g. under the trademark TAXOTERE™, VINBLASTIN R.P.™ and FARMISTIN™, respectively. Discodermolide can be obtained, e.g., as disclosed in U.S. Pat. No. 5,010,099. Epothilones, Including epothilone B and D, and methods for the preparation of such epothilones are in particular generically and specifically disclosed in the patents and patent applications WO 93/10121, U.S. Pat. No. 6,194,181, WO 98/25929, WO 98/08849, WO 99/43653, WO 99/39694, WO 98/22461 and WO 00/31247 in each case in particular in the compound claims and the final products of the working examples. The subject-matter of the final products and the claims comprised by these patents and patent applications is hereby incorporated into the present application by reference to these publications. Comprised are likewise the corresponding stereoisomers as well as the corresponding crystal modifications, e.g. solvates and polymorphs, which are disclosed therein.

The structure of the active agents Identified by code nos., generic or trade names may be taken from the actual edition of the standard compendium “The Merck Index” or from databases, e.g. Patents International (e.g. IMS World Publications). The corresponding content thereof is hereby incorporated by reference.

In analogy to the use of alpha-tubulin acetylation as a biomarker for protein deacetylase inhibition, the present invention therefore also relates to a method of screening a compound for activity against a proliferative disease, which comprises contacting mammalian cells with the compound and detecting an increased level of acetylated alpha-tubulin relative to a control, wherein the compound is a microtubule interacting agent.

The present invention therefore also relates to a method of evaluating a response by a mammalian subject to a microtubule interacting agent, such as especially epothilone B or D, which comprises measuring the level of acetylated alpha-tubulin in cells of the subject and comparing it to the level prior to administration of the microtubule interacting agent.

Accordingly, the present invention also relates to the method of the preceding paragraph wherein measurement of acetylated alpha-tubulin levels takes place ex vivo and to such methods wherein the mammalian subject is a human who has a proliferative disease, such as especially a cancer.

Analogously, the present invention also relates to a method of diagnosing a proliferative disease susceptible to treatment with microtubule interacting agents, such as especially epothilone B or D, in a mammalian subject, which comprises measuring in cells of the subject that exhibits the proliferative disease an increased level of acetylated alpha-tubulin compared to the level prior to administration of the microtubule interacting agent and to such a method wherein measurement of acetylated alpha-tubulin levels takes place ex vivo.

The present invention therefore also provides a method of treating a proliferative disease In a mammalian subject, which comprises administering a microtubule interacting agent to the subject that exhibits the proliferative disease and measuring in cells of the subject an increased level of acetylated alpha-tubulin compared to the level prior to administration of the microtubule interacting agent.

Finally, the present invention also relates to a method of treating a proliferative disease in a mammalian subject, which comprises measuring the level of acetylated alpha-tubulin in cells from the subject that exhibits the proliferative disease and administering a microtubule interacting agent-to the subject if the level of acetylated alpha-tubulin is lower than that exhibited by normal cells of the same type, in particular wherein the microtubule interacting agent is selected from epothilone B or D.

The following examples further illustrate the present invention, and the examples are provided for illustration purposes and are not intended to be limiting the invention thereto.

### EXAMPLE 1

While assessing the effectiveness of HDAC inhibition in tumor cell lines it is observed that in addition to the expected bands of acetylated histones, a protein band migrating at 50 kD appears on immunoblots developed with anti-acetyllysine. For Identifying the protein, cell extracts of HDAC-2 treated and control HCT 166 cells are separated by 2-D gel electrophoresis with a broad range IEF gel (pH 3-10) in the first dimension and analyzed by western blotting with anti-acetyllysine. In the HDAC-2 treated samples, prominently stained bands of low molecular weight corresponding to histones are visible at the lower right corner as well as a non-histone spot around pH 4.5, which is only faintly immunostained in an equivalent analysis of control cell extracts. The position of the non-histone spot suggests that the acetyllysine reactive protein might be tubulin, which is well known to carry this modification.

Acetylation of a lysine residue reduces the charge of a protein because the amide group cannot be protonated. If the protein spot indeed corresponded to alpha-tubulin, the acetylated form is expected to focus at a lower isoelectric point (pI) than its normal counterpart. An IEF-blotting experiment with antibodies against tubulin allows resolving these forms and testing this hypothesis in a one-dimensional analysis on crude extracts. The results indicate that among the antibodies against several tubulin classes, only the antibody against alpha-tubulin showed an HDAC-inhibitor induced acid-shifted band. No shifts are observed with four antibodies against beta-tubulins. This result is corroborated by a 2-dimensional separation on a narrow range pH-gel in the first dimension (pH 4.5-5.5). One set of gels is used for immunoblotting with anti-acetyllysine antibody detection and a second set is reserved for staining of the gel with Coomassie blue. Superposition of the patterns of the antibody stained membrane on the total protein pattern on the membrane indicates that the acetyllysine reactive spot migrates close to the position of alpha-tubulin. A second immunostaining of the same membrane (after removing the first antibody) stains all the area of spots.

The Coomassie stained 2-D gel shows two doublets of spots of similar size in the area of alpha-tubulin for the HDAC-2 treated samples. In untreated cells, the spots at the position adjacent to the main spot of alpha-tubulin are fainter. The 4 tubulin spots, as well as a minor spot which also seemed to become specifically induced by HDAC-2 treatment, are cut and processed for mass spectrometric analysis.

Database searching yields peptides matching the human tubulin alpha-4 chain (Swissprot P04687). Several peptides derived from the acetyllysine-reactive spots match fragments expected to arise from the acetylated form of alpha-tubulin. These are not found in the peptides derived from the non-reactive tubulin isoform. The minor spot, which is barely visible on the Coomassie stained gel, is also identified as alpha-tubulin. It is apparently a fragment of acetylated tubulin.

A commercially available monoclonal antibody specific for the acetylated form of alpha-tubulin (Sigma Aldrich, St. Louis, Mo., USA; Cat #T 6793) further corroborates these conclusions. The signals obtained with this antibody are much stronger than with the general anti-acetyllysine antibody.

### EXAMPLE 2

Time Course of HDAC-2 Induced Tubulin Acetylation in Cell Lines

Acetylation of alpha-tubulin occurs rapidly after addition of HDAC-2. By 30 min after addition of HDAC-2 (200 nM) more than 30% of the maximal acetylation (at 4 hours) is reached. At the last time point (18 hours), cells are visibly damaged.

### EXAMPLE 3

HDAC-2 Increased Tubulin Acetylation in HCT 116 Tumor Xenografts in Mice

In order to correlate the antitumor activity of HDAC-2 with its induction of tubulin acetylation, levels of acetylated tubulin are examined in tumors of athymic mice bearing HCT 116 treated with HDAC-2 and control mice. Mice are dosed with 100 mg/kg of HDAC-2 and the tumors are dissected 3, 6, 16 or 24 h post dose. Tubulin acetylation levels are detected with antibodies specific for acetylated alpha-tubulin. Intravenous administration of HDAC-2 results in consistent increases in acetylated alpha-tubulin within 30 min which lasts for at least 8 h. Proliferating Cell Nuclear Antigen (PCNA) levels are essentially invariant. The increase in tubulin acetylation in HDAC-2 treated animals coupled with the effect of the compound on histone deacetylases indicates that the compounds are inhibiting a protein deacetylase in HCT 116 tumor xenografts.

### EXAMPLE 5

Sensitivity of Tubulin Acetylation to Dose of HDAC-2

To determine the sensitivity of tubulin acetylation to HDAC-2, i.v. doses of 2 mg/kg and 5 mg/kg are administered to HCT 116 tumor bearing mice. Tumors are dissected 1 hour post dose and levels of acetylated tubulin determined by western blotting with the anti-acetylated alpha-tubulin antibody. An i.v. dose as low as 2 mg/kg is able to induce tubulin acetylation in the tumors.

1. A method of screening a compound for activity against a proliferative disease, which comprises contacting mammalian cells with the compound and detecting an increased level of acetylated alpha-tubulin relative to a control.

2. The method of claim 1 wherein the mammalian cells are a cell line established from a subject having a proliferative disease.

3. The method of claim 2 wherein the subject is a human.

4. The method of claim 3 wherein the proliferative disease is a cancer.

5. The method of claim 4 wherein the cancer is a lymphoma, a myeloma, a leukemia, a small cell lung carcinoma, a non-small cell lung carcinoma, an osteosarcoma, a breast carcinoma, a prostrate cancer or a colon cancer.

6. The method of claim 1 wherein the mammalian cells are a contact inhibited mouse fibroblast cell line.

7. The method of claim 2 wherein the cells are in a cell culture.

8. The method of claim 2 wherein the mammalian cells are implanted into a non-human mammalian host.

9. The method of claim 3 wherein the mammalian cells are implanted into a non-human mammalian host.

10. The method of claim 9 wherein the mammalian cells are human cancer cells which are implanted into a rodent.

11. The method of claim 1 wherein the acetylated alpha-tubulin is measured by two-dimensional gel electrophoresis.

12. The method of claim 1 wherein the compound is a protein deacetylase inhibiting compound.

13. The method of claim 12 wherein the protein deacetylase is a histone deacetylase or a tubulin deacetylase.

14. The method of claim 1 wherein the compound is a microtubule interacting agent.

15. A method of evaluating a response by a mammalian subject to a protein deacetylase inhibiting compound which comprises measuring the level of acetylated alpha-tubulin in cells of the subject and comparing it to the level prior to administration of the protein deacetylase inhibiting compound.

16. The method of claim 15 wherein the protein deacetylase inhibiting compound is a histone deacetylase inhibiting compound.

17. The method of claim 16 wherein the histone deacetylase inhibiting compound is selected from N-hydroxy-3-[4-[[[2-(benzofur-3-yl)-ethyl]-amino]methyl]phenyl]-2E-2-propenamide, N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(1H-indol-3-yl)ethyl]-amino]methyl]phenyl]-2E-2-propenamide and N-hydroxy-3-[4-[[[2-(2-methyl-1H-indol-3-yl)-ethyl]-amino]methyl]phenyl]-2E-2-propenamide, or a pharmaceutically acceptable salt thereof.

18. A method of evaluating a response by a mammalian subject to a microtubule interacting agent which comprises measuring the level of acetylated alpha-tubulin in cells of the subject and comparing it to the level prior to administration of the microtubule interacting agent.

19. The method of claim 18, wherein the microtubule interacting agent is epothilone B or D.

20. The method of claim 15 wherein measurement of acetylated alpha-tubulin levels takes place ex vivo.

21. The method of claim 15 wherein the mammalian subject is a human who has a proliferative disease.

22. The method of claim 21 wherein the proliferative disease is a cancer.

23. A method of diagnosing a proliferative disease susceptible to treatment with protein deacetylase inhibiting compounds in a mammalian subject, which comprises measuring in cells of the subject that exhibits the proliferative disease an increased level of acetylated alpha-tubulin compared to the level prior to administration of the protein deacetylase inhibiting compound.

24. The method of claim 23 wherein the protein deacetylase inhibiting compound is a histone deacetylase inhibiting compound.

25. The method of claim 24 wherein the histone deacetylase inhibiting compound is selected from N-hydroxy-3-[4-[[[2-(benzofur-3-yl)-ethyl]-amino]methyl]phenyl]-2E-2-propenamide, N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(1H-indol-3-yl)ethyl]-amino]methyl]phenyl]-2E-2-propenamide and N-hydroxy-3-[4-[[[2-(2-methyl-1H-indol-3-yl)-ethyl]-amino]methyl]phenyl]-2E-2-propenamide, or a pharmaceutically acceptable salt thereof.

26. A method of diagnosing a proliferative disease susceptible to treatment with microtubule interacting agents in a mammalian subject, which comprises measuring in cells of the subject that exhibits the proliferative disease an increased level of acetylated alpha-tubulin compared to the level prior to administration of the microtubule interacting agent.

27. The method of claim 26, wherein the microtubule interacting agent is epothilone B or D.

28. The method of claim 23 wherein measurement of acetylated alpha-tubulin levels takes place ex vivo.

29. A method of treating a proliferative disease in a mammalian subject, which comprises measuring the level of acetylated alpha-tubulin in cells from the subject that exhibit the proliferative disease and administering a protein deacetylase inhibiting compound to the subject if the level of acetylated alpha-tubulin is lower than that exhibited by normal cells of the same type.

30. The method of claim 29 wherein the protein deacetylase inhibiting compound is a histone deacetylase inhibiting compound.

31. The method of claim 30 wherein the histone deacetylase inhibiting compound is selected from N-hydroxy-3-[4-[[[2-(benzofur-3-yl)-ethyl]-amino]methyl]phenyl]-2E-2-propenamide, N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(1H-indol-3-yl)ethyl]-amino]methyl]phenyl]-2E-2-propenamide and N-hydroxy-3-[4-[[[2-(2-methyl-1H-indol-3-yl)-ethyl]-amino]methyl]phenyl]-2E-2-propenamide, or a pharmaceutically acceptable salt thereof.

32. A method of treating a proliferative disease in a mammalian subject, which comprises measuring the level of acetylated alpha-tubulin in cells from the subject that exhibits the proliferative disease and administering a microtubule interacting agent to the subject if the level of acetylated alpha-tubulin is lower than that exhibited by normal cells of the same type.

33. The method of claim 32 wherein the microtubule interacting agent is epothilone B or D.

34. Use of alpha-tubulin acetylation as a biomarker for protein deacetylase inhibition.

35. Use of alpha-tubulin acetylation as a biomarker for the activity of microtubule interacting agents.
